(19) United States
(12) Patent Application Publication
(10) Pub. No.: US 2024/0215965 A1
(43) Pub. Date: Jul. 4, 2024

(54) ENDOSCOPIC SURGERY IRRIGATION DEVICE ALLOWING A CONTINUOUS FEED

(71) Applicant: Abderrazik CHIKHI, Bejaia (DZ)

(72) Inventor: Abderrazik CHIKHI, Bejaia (DZ)

(21) Appl. No.: 18/265,828

(22) PCT Filed: Nov. 28, 2021
(86) PCT No.: PCT/DZ2021/050004

§ 371 Date: Mar. 4, 2024

(51) Int. Cl. A61B 17/00

(52) U.S. Cl. CPC A61B 17/00234; A61B 2017/00296; A61B 2217/007

(57) ABSTRACT

The endoscopic surgery device that we have equipped with an intermediate storage pocket, allows us to achieve a surgical procedure without interruption nor disruption.

This storage pocket insures a permanent irrigation which allows a better distension and a better visibility in order to make the surgery procedure easier.

This continuous solutes supply prevents the air from entering inside the tubing.

In fact the air that used to enter in the former device, constitutes a major constraint for surgeons who have to interrupt their intervention and see the surgical procedure, passing from few minutes to hours.

### FIELD OF INVENTION

This invention concerns the area of endoscopic surgery related to: Gynecological surgery: resecting fibromas, polyps, uterine malformation (partition, etc); Urological surgery: Resection of bladder tumors, prostate adenomas, etc; and Arthroscopic Surgery: Rotators cuff, Cuff tendons repair, acromioplasty, ligament surgery, etc.

### DESCRIPTION OF RELATED ART

It is known that this technique of endoscopic surgery is based on a resectoscope or an endoscope connected to a camera, supplied by a bipolar or monopolar energy. Equipped with two taps, an upper inlet for solutes supply and a lower outlet for the draining, this latter is connected to a suction bottle. This resector is also connected to an irrigation line, connected to a surgical pump (Hystero Pump-Arthro-pump), this latter is aimed to supply permanently, the organ that will bear the surgery (Uterus, Bladder, Articulations).

This pump sucks solutes through an irrigation line shaped as “Y” from two hanging Bottles. The first objective of this device is to dilate the organ for a better practical surgery procedure and a better visualization for the surgeon.

This alternated alimentation performed by the operating room nurse, causes inconveniences, practically speaking. In fact, the operation nurse is called to unplug the supplying line, every time a bottle is emptied in order to connect a new one and this happens for several times. In fact, during this handlings some air bubbles enter inside the pipes and firstly, this can cause damages to the pump, because of the sudden change in the regime as the surgical pump will run on empty, but also and all the more that the surgery procedure became difficult and sometimes even impossible.

The air that enters causes a bad visibility which requires from the surgeon to stop and withdraw his working tool in order to expel the air. This constraint is often followed by an acute bleeding, due to the lack of supply of the used solutes. These disruptions extend the surgeries and the anesthesia time, causing a major risk for patients.

There is also losses on the economical level, by reason of the abusive use of solutes and of anesthesia's products.

### INVENTION'S PURPOSE

This purpose of this invention is to overcome the above mentioned disadvantages, in order to perform a comfortable, rapid and without difficulties surgery procedure and with a minimum use of solutes. This invention allows us also, to minimize the duration of the surgical procedure in a significant manner and in the same time to avoid the anesthesia risks.

This device that ensures a continuous supply with solutes, allows the protection of the pump from eventual damages, thanks to its permanent connection to the storage pump, from the beginning until the end of the surgery and avoids air infiltration inside the operation area that causes major risks for the patients.

### DESCRIPTION OF FIGURES

FIG. 1: Intermediary storage pocket with irrigation lines.

FIG. 2: Overview of the device connected to an endoscope

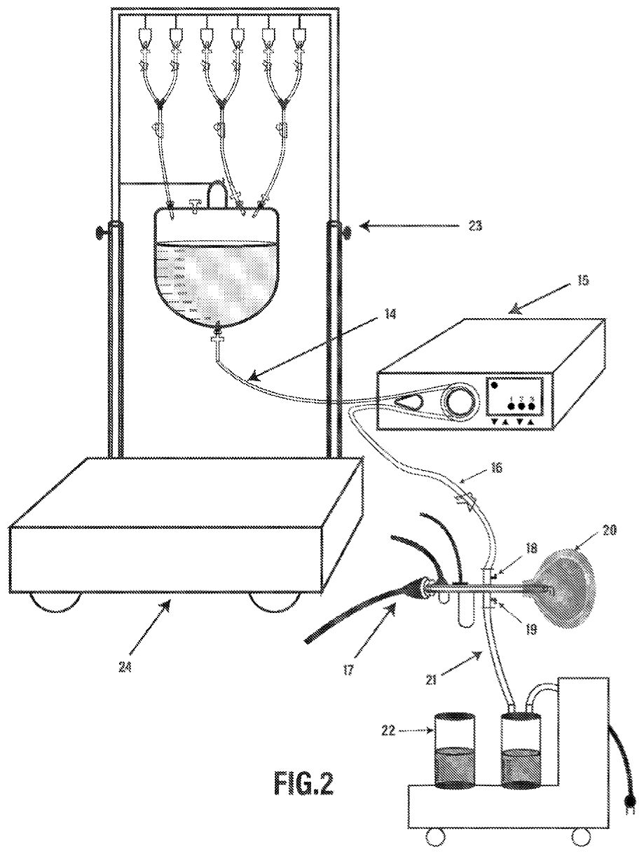

### DETAILED DESCRIPTION

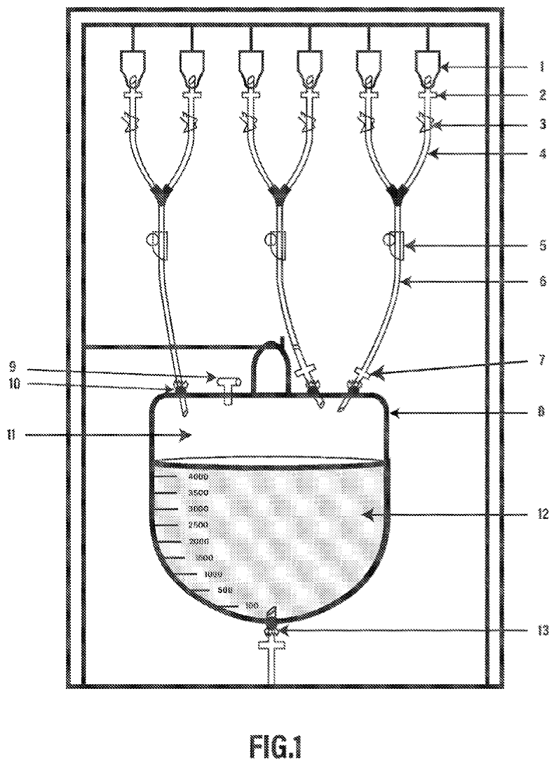

This invention consists in ensuring a solutes irrigation in a continuous way, by providing a storage pocket of 8 intermediaries, empty, packed, translucent, sterile, medical interior/exterior, for a single use and of a capacity of 5000 cc (See FIG. 1).

The above mentioned pocket 8, equipped with an expelling air opening 9 which upper part is also connected to a water resistant opening 13, closed by an irrigation line 14 that is connected to the surgical pump (Hystero pump, Arthro pump) 15 in a permanent way, during the operation. This storage pocket of 5000 cc, divided into two spaces, space 12 of ⅘ of its capacity which is kept for solutes, in the same time it keeps the same quantity from the beginning until the end of the operation and a space 11 of ⅕ of its capacity kept for air. This pocket is supplied by several bottles, 1 through irrigating lines 4 in “Y” shape, packed with that pocket in a sterilized packaging, with extremities supplied with perforated trocars 2 et 7 in order to make their introduction inside the storage pocket 8 easier and the supply through water resistant, closed and integrated caps 10.

The supplying lines 6 of the so called storage pocket 8 are specific. They are flexible, with an “Y” shape equipped with clumps for Start and Stop 3 for the two superior lines 4 and of debit regulating wheels 5 for lower lines 6 that are connected to the storage pocket 8.

This debit is controlled, according to the needs of the performed surgery and this, in order to first, avoid solutes supplying interruptions which was frequent in the previous irrigation system, and from another part, to avoid the solutes losses.

This new device allows us to work with various available volumes bottles 1 and with any kind of solutes that are considered useful by the endoscopy (Physiological Serum, Glycol) and avoiding major risks for the patients, because of the frequent air infiltration within the former system.

Filling the so called pocket 8, is performed at the same time with the surgical operation in a sterile packaging in the operation room. This device is adaptable to all the endoscopic irrigation systems.

This device is hanged to a variable height crane 23, along with a mobile platform 24 to make the task of the operating room nurse easier, thanks to several pockets, without haste, while respecting notions of sterilization, at the same time. It also facilitate the endoscopy performer job in an area of ideal distension for a better act practicing.

The so called surgical pump (Histero pump, arthropump) 15, injects solutes in the work tool 17, through a the line 16, towards the organ 20 that is to be operated. (Uterus, Bladder, Articulation), through an upper inlet tap 18. The draining is performed by a line 21, connected to the aspiration bottle 22, through the lower tap 19.

1. Irrigation device for the endoscopic surgery, with a sterile medical solute storage pocket, medicalized, sterile Inside/Outside, allowing a continuous supply, characterized by the fact that the internal intermediate solutes storage pocket (8), consists of four essential elements, for a single use (disposable) and an empty translucent pocket, packaged with its three Y lines, in a sterile packaging, graduated with a capacity of “5000 cc”, divided into two spaces ⅘ of its capacity and is kept for the permanent storage and solutes circulation, while keeping the same quantity from the beginning until this end of surgeries and ⅕ of its capacity remains empty and kept for the air circulation. On the upper part of the so called pocket (8) through an opening (9) in order to get rid of it and prevent it from entering inside the operation area.

2. Device according to the claim 1, characterized by the supply lines (6) in “′Y” shape, equipped with clumps Start/Stop (3) debit regulating wheels (5) and perforated trocars on the two extremities (2, 7) in order to facilitate their introduction within the storage pocket (8), wherein the two upper extremities (4) are connected to solutes bottles (1) and hanged above.

3. Device according to the claim 1 characterized by the fact that caps (10), are closed, water resistant on the upper part of the pocket (8), they allow the supply lines connection (6), a water resistant opening (13), situated in the lower part of the pocket, allows the continuous solutes outlet, in order to supply the endoscopy performer tool, with a single plugging total and final.
